(19) United States
(12) Patent Application Publication
(10) Pub. No.: US 2009/0241645 A1
(43) Pub. Date: Oct. 1, 2009

(54) SYSTEM AND METHOD FOR DETERMINING TRANSMISSION OIL TYPE

(76) Inventors: Mark A. Rains, Indianapolis, IN (US); Randall S. Conn, Indianapolis, IN (US)

Correspondence Address: ALLISON TRANSMISSION, INC., BARNES & THORNBURG LLP, 11 SOUTH MERIDIAN STREET, INDIANAPOLIS, IN, 46204, US

(21) Appl. No.: 12/059,409

(22) Filed: Mar. 31, 2008

(51) Int. Cl. G01N 33/26; F01M 1/18

(52) U.S. Cl. 73/53.05; 184/6.4

(57) ABSTRACT

Transmission oil type may be determined based on the opening time of a transmission latch valve and on oil temperature. An opening time of a latch valve positioned between an oil reservoir and one or more torque transmitting devices of a transmission is determined in response to a command to open the latch valve. A temperature of oil supplied by the latch valve from the oil reservoir to the one or more torque transmitting devices in response to the command to open the latch valve is also determined. The latch valve opening time and oil temperature are monitored over a range of oil temperatures to collect opening times of the latch valve at each of a plurality of oil temperatures within the range of oil temperatures. The transmission oil type is then determined based on the opening times of the latch valve at each of the plurality of oil temperatures.

### FIELD OF THE INVENTION

The present invention relates generally to transmissions for motor vehicles, and more specifically to systems and methods for determining and identifying the type of oil being used in a transmission.

### BACKGROUND

Conventional automatic transmissions for use with motor vehicles typically contain transmission oil that is used to lubricate components and to provide hydraulic pressure for controlling operation of one or more torque transmitting apparatuses, e.g., clutches. It is desirable to be able to non-invasively test the transmission oil to determine, if possible, its type.

### SUMMARY

The present invention may comprise one or more of the features recited in the attached claims, and/or one or more of the following features and combinations thereof. A system for determining transmission oil type may comprise a transmission including a latch valve positioned between an oil reservoir containing transmission oil and one or more torque transmitting devices, an actuator configured to be responsive to an actuator control signal to open the latch valve to supply oil from the oil reservoir to the one or more torque transmitting devices, a first sensor configured to produce a sensor signal from which an opening time of the latch valve can be determined, a second sensor configured to produce a temperature signal corresponding to a temperature of the oil supplied by the latch valve to the one or more torque transmitting devices, and a control circuit. The control circuit may include a memory having instructions stored therein that are executable by the control circuit to process the sensor and temperature signals following production of the actuator control signal over a range of oil temperatures to collect opening times of the latch valve at each of a plurality of temperatures within the range of oil temperatures, and to then determine the transmission oil type based on the opening times of the latch valve at each of the plurality of temperatures.

The system may further comprise an oil pump positioned between the latch valve and the oil reservoir. The oil pump may be configured to be responsive to a pump control signal to supply oil from the oil reservoir to the latch valve. The control circuit may be configured to produce the pump control signal. The latch valve may define an inlet fluidly coupled to the oil pump and an outlet fluidly coupled to the one or more torque transmitting devices. The first sensor may comprise a pressure sensor configured to produce a pressure signal corresponding to a pressure of the oil exiting the outlet of the latch valve. In this embodiment, the instructions stored in the memory may further include instructions that are executable by the control circuit to monitor the pressure signal following production of the actuator control signal and to determine that the latch valve is open when the pressure of the oil exiting the outlet of the latch valve reaches a predefined pressure value. Alternatively, the first sensor may comprise a position sensor configured to produce a position signal corresponding to a position of the latch valve relative to a reference position. In this embodiment, the instructions stored in the memory may further include instructions that are executable by the control circuit to monitor the position signal following production of the actuator control signal and to determine that the latch valve is open when the position of the latch valve relative to the reference position reaches a predefined position.

The instructions stored in the memory may further include instructions that are executable by the control circuit to process the sensor and temperature signals following production of the actuator control signal over the range of oil temperatures to collect a plurality of opening times of the latch valve at each of a plurality of temperatures within the range of oil temperatures, to process the plurality of opening times of the latch valve at each of the plurality of temperatures to determine an average opening time of the latch valve at each of the plurality of temperatures, and to then determine the transmission oil type based on the average opening times of the latch valve at each of the plurality of temperatures.

The system may further comprise a transmission oil map stored in memory. The transmission oil map may define expected opening time values and corresponding opening time variance values at each of the plurality of temperatures for a predefined transmission oil type. The instructions stored in the memory may further include instructions that are executable by the control circuit to compare the opening times of the latch valve to the expected opening time values and corresponding opening time variance values at each of the plurality of temperatures, and to determine that the transmission oil is the predefined transmission oil type if differences between at least a predefined number of the opening times of the latch valve match corresponding ones of the expected opening time values within the corresponding opening time variance values.

The system may further comprise a plurality of transmission oil maps stored in memory. Each of the plurality of transmission oil maps may define expected opening time values and corresponding opening time variance values at each of the plurality of temperatures for a different transmission oil type. The instructions stored in the memory further include instructions that are executable by the control circuit to compare the opening times of the latch valve to the expected opening time values and corresponding opening time variance values at each of the plurality of temperatures for each of the plurality of transmission oil maps, and to determine that the transmission oil is a transmission oil type defined by one of the plurality of transmission oil maps if differences between at least a predefined number of the opening times of the latch valve match corresponding ones of the expected opening time values of the one of the plurality of transmission oil maps within the corresponding opening time variance values.

A method for determining transmission oil type may comprise opening a latch valve positioned between an oil reservoir and one or more torque transmitting devices of a transmission to supply transmission oil from the reservoir to the one or more torque transmitting devices, determining an opening time of the latch valve, determining a temperature of the oil supplied by the latch valve to the one or more torque transmitting devices in response to opening the latch valve, repeatedly opening the latch valve, determining an opening time of the latch valve and determining a temperature of the oil supplied by the latch valve over a range of oil temperatures to collect opening times of the latch valve at each of a plurality of oil temperatures within the range of oil temperatures, determining the transmission oil type based on the opening times of the latch valve at each of the plurality of oil temperatures, and storing the transmission oil type in memory.

The method may further comprise collecting a plurality of opening times of the latch valve at each of a plurality of temperatures within the range of oil temperatures, and processing the plurality of opening times of the latch valve at each of the plurality of temperatures to determine an average opening time of the latch valve at each of the plurality of temperatures. Determining the transmission oil type may then comprise determining the transmission oil type based on the average opening times of the latch valve at each of the plurality of temperatures.

The method may further comprise comparing the opening times of the latch valve to expected opening time values and corresponding opening time variance values for a predefined transmission oil type at each of the plurality of temperatures. Determining the transmission oil type may then comprise determining that the transmission oil is the predefined transmission oil type if differences between at least a predefined number of the opening times of the latch valve match corresponding ones of the expected opening time values within the corresponding opening time variance values.

The method may further comprise comparing the opening times of the latch valve to expected opening time values and corresponding opening time variance values at each of the plurality of temperatures for each of a plurality of different transmission oil types. Determining the transmission oil type may then comprise determining that the transmission oil is a transmission oil type defined by one of the plurality of different transmission oil maps if differences between at least a predefined number of the opening times of the latch valve match corresponding ones of the expected opening time values of the one of the plurality of different transmission oil maps within the corresponding opening time variance values.

The method may further comprise storing an undetermined oil type indicator in memory if the transmission oil type cannot be determined based on the opening times of the latch valve at each of the plurality of oil temperatures.

The method may further comprise activating a warning indicator if the transmission oil type cannot be determined based on the opening times of the latch valve at each of the plurality of oil temperatures.

The method may further comprise, if the transmission oil type cannot be determined based on the opening times of the latch valve at each of the plurality of oil temperatures, logging at least one of miles traveled by a vehicle carrying the transmission and operating time of the transmission following determination that the transmission oil type cannot be determined.

The method may further comprise modifying an oil change scheduling process if the transmission oil type cannot be determined based on the opening times of the latch valve at each of the plurality of oil temperatures.

The method may further comprise activating a warning indicator if the transmission oil type is determined, based on the opening times of the latch valve at each of the plurality of oil temperatures, to be an undesirable transmission oil type.

The method may further comprise, if the transmission oil type is determined based on the opening times of the latch valve at each of the plurality of oil temperatures to be an undesirable transmission oil type, logging at least one of miles traveled by a vehicle carrying the transmission and operating time of the transmission following determination that the transmission oil type is undesirable.

The method may further comprise modifying an oil change scheduling process if the transmission oil type is determined, based on the opening times of the latch valve at each of the plurality of oil temperatures, to be an undesirable transmission oil type.

A method for determining transmission oil type may comprise determining an opening time of a latch valve positioned between an oil reservoir and one or more torque transmitting devices of a transmission in response to a command to open the latch valve, determining a temperature of oil supplied by the latch valve from the oil reservoir to the one or more torque transmitting devices in response to the command to open the latch valve, repeating determining an opening time of the latch valve and determining a temperature of oil supplied by the latch valve over a range of oil temperatures to collect opening times of the latch valve at each of a plurality of oil temperatures within the range of oil temperatures, determining the transmission oil type based on the opening times of the latch valve at each of the plurality of oil temperatures, and storing the transmission oil type in memory.

### BRIEF DESCRIPTION OF THE DRAWINGS

FIG. 1 is a block diagram of one illustrative embodiment of a system for determining the type of oil being used in a motor vehicle transmission.

FIG. 2 is a flowchart of one illustrative embodiment of a process for determining the type of oil being used in a motor vehicle transmission.

FIG. 3 is a flowchart of one illustrative embodiment of a transmission oil type determination routine used by the process of FIG. 2.

FIG. 4 is a flowchart of one illustrative embodiment of a latch valve opening time averaging routine used by the routine of FIG. 3.

FIGS. 5A and 5B comprise a flowchart of one illustrative embodiment of a latch valve opening time and oil temperature processing routine used by the routine of FIG. 3.

### DESCRIPTION OF THE ILLUSTRATIVE EMBODIMENTS

For the purposes of promoting an understanding of the principles of the invention, reference will now be made to a number of illustrative embodiments shown in the attached drawings and specific language will be used to describe the same.

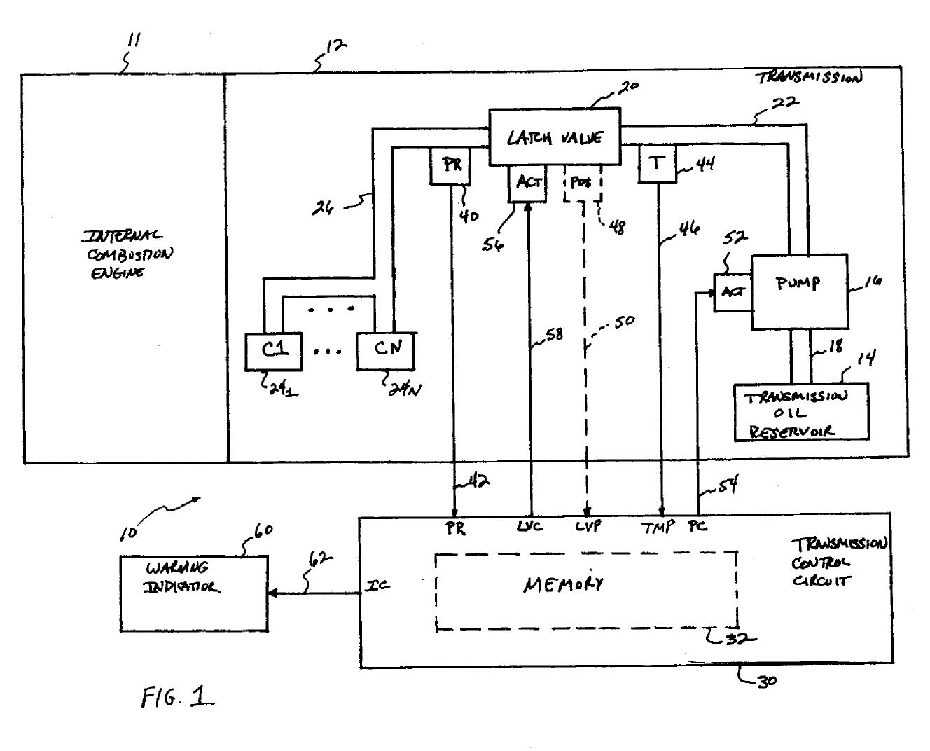

Referring now to FIG. 1, a block diagram of one illustrative embodiment of a system 10 for determining the type of oil being used in a motor vehicle transmission 12 is shown. In the illustrated embodiment, the transmission 12 is operatively coupled to an internal combustion engine 11. The transmission 12 is illustratively a conventional automatic transmission, i.e., a transmission having a number of automatically selectable gear ratios. As it relates to this disclosure, the transmission 12 includes a transmission oil reservoir or sump 14 that is configured to hold a quantity of transmission oil. The transmission oil reservoir 14 is coupled via a conduit 18 to an input of a conventional oil pump 16 having an output that is coupled via a conduit 22 to an inlet of a conventional latch valve 20. An output of the latch valve 20 is fluidly coupled to a number of conventional torque transmitting devices, 24₁-24_{N}, via a conduit 26, wherein N may be any positive integer. The number of torque transmitting devices, 24₁-24_{N}, may be, for example, conventional clutches, although other torque transmitting devices are contemplated. The system 10 further includes a transmission control circuit 30 that is configured to manage and control the overall operation of the transmission 12. Illustratively, the transmission control circuit 30 is microprocessor-based, and includes a memory unit 32 having instructions stored therein that are executable by the control circuit 30 to control operation of the transmission 12.

The transmission 12 includes a number of sensors or sensing systems each configured to supply a sensor signal to the control circuit 30 that corresponds to an operating parameter of the transmission 12. For example, the transmission 12 may illustratively include a pressure sensor 40 that is fluidly coupled to the outlet of the latch valve 20, and that is electrically connected to a pressure input, PR, of the transmission control circuit 30 via a signal path 42. The pressure sensor 40 may be conventional, and is operable to produce a pressure signal that corresponds to the fluid pressure at the outlet of the latch valve 20, which thus corresponds to the pressure of transmission oil supplied by the latch valve 20 to the torque transmitting devices 24₁-24_{N}. The transmission 12 further includes a temperature sensor 44 that is fluidly coupled to the inlet of the latch valve 20, and that is electrically connected to an oil temperature input, TMP, of the transmission control circuit 30 via a signal path 46. It will be understood that while the temperature sensor 44 is shown as being fluidly coupled to the conduit 22 adjacent to the inlet of the latch valve 20, the temperature sensor 44 may alternatively be located anywhere along the conduit 22, the conduit 18 or on, or in fluid communication with, the transmission oil reservoir 14 In any case, the temperature signal 44 may be conventional, and is operable to produce a temperature signal corresponding to the temperature of the transmission oil supplied by the latch valve 20 to the one or more torque transmitting devices 24₁-24_{N}.

Illustratively, as shown in phantom in FIG. 1, the transmission 12 may further include a position sensor 48 that is operatively coupled to the latch valve 20, and that is electrically connected to a latch valve position input, LVP, of the transmission control circuit 30 via a signal path 50. In embodiments that include such a position sensor 48, the sensor 48 is operable to produce a position signal that corresponds to an operating position of the latch valve 20 relative to a reference position of the valve 20. The reference position of the latch valve 20 illustratively corresponds to a fully closed position that prevents oil from passing through the latch valve 20 from the pump 16 to one or more the torque transmitting devices 24₁-24_{N}.

The transmission 12 further includes a number of actuators that are responsive to control signals produced by the transmission control circuit 30 to control various operational aspects of the transmission 12. For example, the transmission 12 includes a conventional pump actuator 52 that is electrically connected to a pump control output, PC, of the transmission control circuit 30 via a signal path 54. The pump actuator 52 is responsive to the pump control signals produced by the transmission control circuit 30 to control operation of the oil pump 16 in a conventional manner. The transmission 12 further includes a latch valve actuator 56 that is coupled to the latch valve 20, and that is electrically to a latch valve control output, LVC, of the transmission control circuit 30 via a signal control path 58. The latch valve actuator 56 may be provided in the form of a conventional solenoid or other conventional actuator that is responsive to the latch valve control signal produced by the transmission control circuit 30 to control the position of the latch valve 20 relative to its reference position. It will be understood, in embodiments that include the position sensor 48, that while FIG. 1 illustrates the position sensor 48 as being coupled to the latch valve 20, the position sensor 48 may alternatively be coupled to the actuator 56 such that the position signal produced by the position signal 48 corresponds to a position of the actuator 56 relative to a reference actuator position.

The system 10 further includes a warning indicator 60 that is electrically connected to an indicator control output, IC, of the transmission control circuit 30 via a signal path 62. The warning indicator 60 may be a conventional visual and/or audible indicator that may be actuated or activated by the transmission control circuit 30 to provide an operator and/or occupant of a vehicle carrying the engine 11 and transmission 12 with notification of a warning condition associated with the transmission 12.

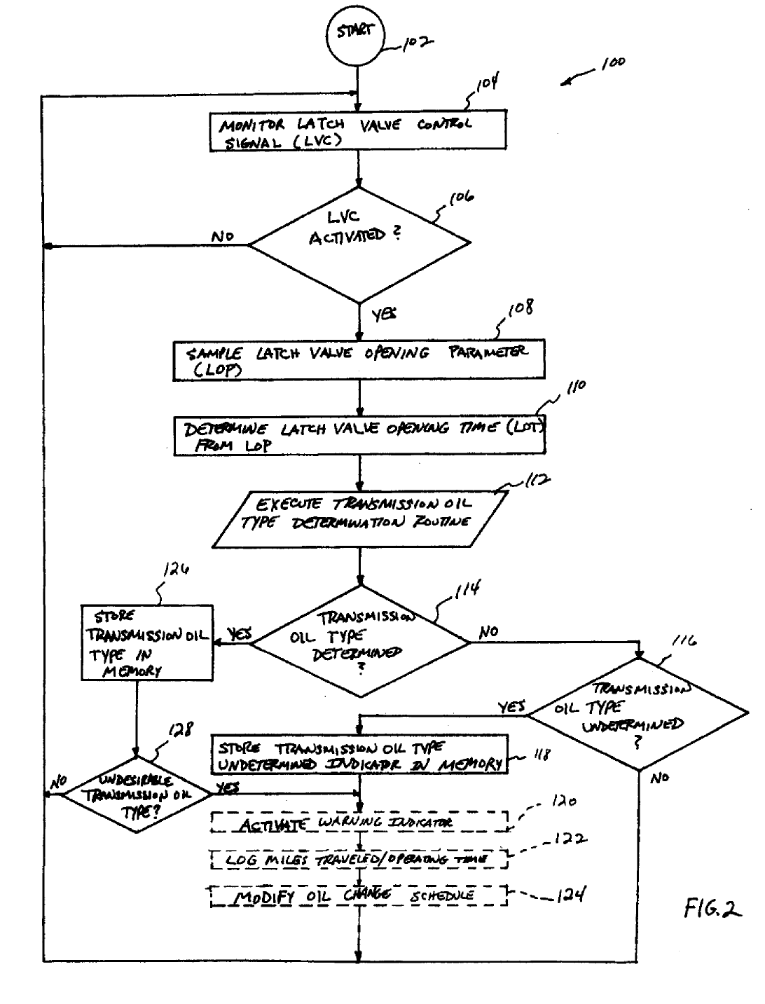

Referring now to FIG. 2, a flow chart of one illustrative embodiment of a process 100 for determining the type of oil being used in the transmission 12, i.e., carried within the oil reservoir 14, is shown. Illustratively, the process 100 is stored in the memory unit 32 in the form of instructions that are executable by the control circuit 30 to determine the type of oil being used in the transmission 12. The process 100 begins at step 102, and thereafter at step 104 the control circuit 30 is operable to monitor the latch valve control signal, LVC, that is produced by the control circuit 30. Thereafter at step 106, the control circuit 30 is operable to determine whether the latch valve control signal, LVC, is being, or is about to be, activated. If not, the process 100 loops back to step 104.

If, at step 106, the control circuit 30 determines, by monitoring the latch valve control signal, LVC, that the latch valve actuator 56 is being, or is about to be, activated to open the latch valve 20, the process 100 advances to step 108 where the control circuit 30 is operable to sample a latch valve opening parameter, LOP. Thereafter at step 110, the control circuit 30 is operable to determine from the latch valve opening parameter, LOP, monitored at step 108, a latch valve opening time, LOT. The latch valve opening time, LOT, corresponds to a time duration, or an elapsed time, between activation of, or commanding, the latch valve 20 from a fully closed to an open position.

In one embodiment, the latch valve opening parameter, LOP, corresponds to the pressure signal produced by the pressure sensor 40. In this embodiment, the instructions stored in the memory unit 32 include instructions that are executable by the transmission control circuit 30 to process the pressure signal produced by the pressure sensor 40 to determine the latch valve opening time, LOT. Illustratively, the control circuit 30 is operable to determine the latch valve opening time, LOT, from the pressure signal produced by the pressure sensor 40 by monitoring the pressure signal produced by the pressure signal 40 when the latch valve 20 is activated or commanded to open, as determined by monitoring the activation of the latch valve command, LVC, and to then determine that the latch valve 20 is open when the fluid pressure at the outlet of the latch valve 20 has reached, or exceeds, a predefined pressure value. In this embodiment, the elapsed time between activation or opening of the latch valve 20 and the fluid pressure at the outlet of the latch valve 20 reaching or exceeding the predefined pressure value is the latch valve opening time, LOT.

In an alternative embodiment in which the transmission 12 includes the position sensor 48, the latch valve opening parameter, LOP, that is monitored at step 108 is the position signal produced by the position sensor 48. In this embodiment, the transmission control circuit 30 is operable to monitor the position signal produced by the position sensor 48, and to determine the latch valve opening time, LOT, as the duration or elapsed time between the activation of the latch valve command, LVC, and a subsequent predefined position of the latch valve 20 relative to its reference position.

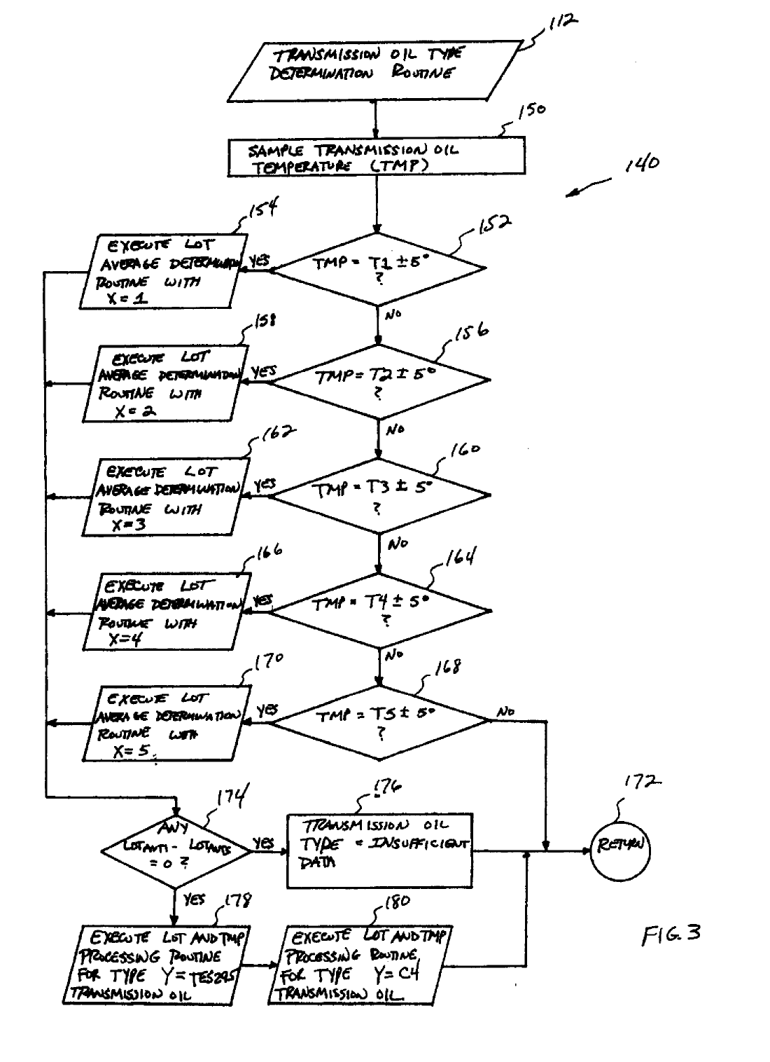

The process 100 advances from step 110 to step 112 where a transmission oil type determination routine is executed. As will be described in greater detail hereinafter with respect to FIG. 3, the control circuit 30 is operable to execute the transmission oil type determination routine to determine a value that corresponds to the type of oil in the transmission 12, a value that indicates that the type of oil in the transmission 12 is undetermined or a value that indicates that insufficient data is available to determine the type of transmission oil in the transmission 12. Following step 112, the process 100 advances to step 114 where the control circuit 30 is operable to determine whether a transmission oil type was determined by the transmission oil type determination routine at step 112. If not, the process 100 advances to step 116 where the control circuit 30 determines whether a transmission oil type was undetermined. If not, insufficient data was available at the time the transmission oil type determination routine was executed to determine the type of oil in the transmission 12, and the process 100 loops back to step 104 to collect additional data relating to the operation of the latch valve 20.

If, at step 116, the control circuit 30 determines that the transmission oil type was undetermined by the transmission oil type determination routine at step 112, the process 100 advances to step 118 where the control circuit 30 is operable to store a “transmission oil type undetermined” indicator in the memory unit 32. Illustratively, the transmission oil type undetermined indicator may be stored in the memory unit 32 in the form of an error code, a flag, a suitable message, or the like. As shown by dashed-line representation, the process 100 may or may not include additional steps 120, 122 and/or 124. If step 120 is included, the control circuit 30 is operable at step 120 to activate the warning indicator 60 to thereby notify the vehicle operator that the transmission oil type was undetermined or cannot be determined. If step 122 is included, the control circuit 30 is operable at step 122 to begin logging the miles traveled and/or to begin logging the operating time of the transmission 12 subsequent to determining that the transmission oil type could not be determined by the transmission oil type determination routine at step 112. If step 124 is included in the process 100, the transmission control circuit 30 is operable at step 124 to modify a conventional oil change schedule process that is being executed and monitored by the control circuit 30. For example, the transmission control circuit 30 may be operable at step 124 to modify the oil change schedule such that oil changes occur more frequently when the transmission oil type is undetermined than would otherwise be the case when the transmission oil type is determined to be an acceptable oil type. As another example, the transmission control circuit 30 may be operable at step 124 to modify the oil change schedule so as to alert the vehicle operator to change the transmission oil immediately or at the next convenient time. In any case, the process 100 loops from any of steps 118, 120, 122 and 124, depending upon whether and which of any of the steps 120, 122 or 124 are included in the process 100, to step 104.

If, at step 114, the control circuit 30 determines that the transmission oil type was determined by the transmission oil type determination routine at step 112, the process 100 advances to step 126 where the control circuit 30 is operable to store the transmission oil type in the memory unit 32. Illustratively, the control circuit 30 may store the transmission oil type in the memory unit 32 in the form of a code, suitable message or the like. Following step 126, the process 100 advances to step 128 where the control circuit 30 is operable to determine whether the transmission oil type determined by the transmission oil type determination routine at step 112 is an undesirable or unacceptable transmission oil type. If so, the process 100 may advance to any of steps 120, 122 or 124 depending on whether and which of any of the steps 120, 122 or 124 are included in the process 100. If, at step 128, the control circuit 30 instead determines that the transmission oil type determined by the transmission oil type determination routine at step 112 is not an undesirable or unacceptable transmission oil type, the process 100 loops back to step 104. Step 128 is illustratively included in the process 100 to address embodiments in which the transmission oil type determination routine executed at step 112 is able to determine the type of oil in the transmission 12, but that the type of oil detected is an undesirable or unacceptable oil type for the particular implementation of the transmission 12.

Referring now to FIG. 3, a flow chart is shown of one illustrative embodiment 140 of the transmission oil type determination routine executed by the process 100 at step 112. The transmission oil type determination routine 140 begins at step 150 where the control circuit 30 is operable to sample the transmission oil temperature, TMP, produced by the temperature sensor 44. Thereafter at step 152, the transmission control circuit 30 is operable in one illustrative embodiment to determine whether the sampled transmission oil temperature, TMP, is equal to a first temperature, T1, within plus or minus 5°, e.g., degrees Celsius. Alternatively, the control circuit 30 may be operable at step 152 to determine whether the sampled transmission oil temperature, TMP, is equal to the first temperature, T1, within plus or minus J°, where J may be any positive integer or positive real number, and where the temperature may be expressed in units other than degrees Celsius. In any case, the “YES” branch of step 152 advances to step 154 where the control circuit 30 is operable to execute a LOT (latch valve opening time) average determination routine with a temperature range identification variable X equal to 1. The “NO” branch of step 152 advances to step 156.

At step 156, the control circuit 30 is operable in one illustrative embodiment to determine whether the sampled transmission oil temperature, TMP, is equal to a second temperature, T2, within plus or minus 50, e.g., degrees Celsius. Alternatively, the control circuit 30 may be operable at step 156 to determine whether the sampled transmission oil temperature, TMP, is equal to the second temperature, T2, within plus or minus J°, where J may be any positive integer or positive real number, and where the temperature may be expressed in units other than degrees Celsius. In any case, the “YES” branch of step 156 advances to step 158 where the control circuit 30 is operable to execute the LOT (latch valve opening time) average determination routine with the temperature range identification variable X equal to 2. The “NO” branch of step 156 advances to step 160.

At step 160, the control circuit 30 is operable in one illustrative embodiment to determine whether the sampled transmission oil temperature, TMP, is equal to a third temperature, T3, within plus or minus 5°, e.g., degrees Celsius. Alternatively, the control circuit 30 may be operable at step 160 to determine whether the sampled transmission oil temperature, TMP, is equal to the third temperature, T3, within plus or minus JO, where J may be any positive integer or positive real number, and where the temperature may be expressed in units other than degrees Celsius. In any case, the “YES” branch of step 160 advances to step 162 where the control circuit 30 is operable to execute the LOT (latch valve opening time) average determination routine with the temperature range identification variable X equal to 3. The “NO” branch of step 160 advances to step 164.

At step 164, the control circuit 30 is operable in one illustrative embodiment to determine whether the sampled transmission oil temperature, TMP, is equal to a fourth temperature, T4, within plus or minus 5°, e.g., degrees Celsius. Alternatively, the control circuit 30 may be operable at step 164 to determine whether the sampled transmission oil temperature, TMP, is equal to the fourth temperature, T4, within plus or minus J°, where J may be any positive integer or positive real number, and where the temperature may be expressed in units other than degrees Celsius. In any case, the “YES” branch of step 164 advances to step 166 where the control circuit 30 is operable to execute the LOT (latch valve opening time) average determination routine with the temperature range identification variable X equal to 4. The “NO” branch of step 164 advances to step 168.

At step 168, the control circuit 30 is operable in one illustrative embodiment to determine whether the sampled transmission oil temperature, TMP, is equal to a fifth temperature, T5, within plus or minus 50, e.g., degrees Celsius. Alternatively, the control circuit 30 may be operable at step 168 to determine whether the sampled transmission oil temperature, TMP, is equal to the fifth temperature, T5, within plus or minus JO, where J may be any positive integer or positive real number, and where the temperature may be expressed in units other than degrees Celsius. In any case, the “YES” branch of step 168 advances to step 170 where the control circuit 30 is operable to execute the LOT (latch valve opening time) average determination routine with the temperature range identification variable X equal to 5. The “NO” branch of step 168 advances to step 172 where the transmission oil type determination routine 112 returns to the process 100 to FIG. 2.

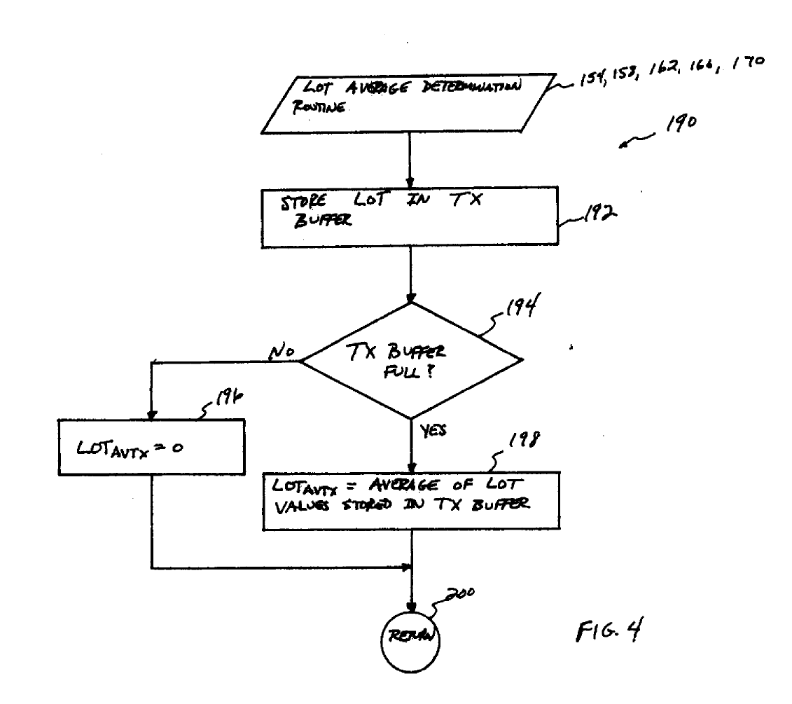

In the embodiment of the routine 140 illustrated in FIG. 4, the latch valve opening times are collected and processed in five separate and different temperature groups each defined by one of five corresponding base temperatures T1-T5. In one example embodiment, T1=10° C., T2=200° C., T3=300° C., T4=400° C. and T5=500° C., although the values of T1-T5 may alternatively be set to other temperature values within the same, smaller or larger temperature range. Alternatively still, more or fewer base temperatures may be used, and/or the temperature ranges defined by the base temperatures and the corresponding J° variances may be overlapping or non-overlapping.

Following execution of the LOT average determination routine at of the steps 154, 158, 162, 166 and 170, the routine 140 advances to step 174 where the control circuit 30 is operable to determine whether any of the latch opening time average values, LOT_{AVT1}-LOT_{AVT5}, are equal to zero. If so, the routine 140 advances to step 176 where the control circuit 30 is operable to set the transmission oil type to an insufficient data indicator. Thereafter, the routine advances to step 172 where the routine 140 is returned to the process 100 of FIG. 2.

If, at step 174, the control circuit 30 determines that none of the latch opening time average values, LOT_{AVT1}-LOT_{AVT5}, are equal to zero, the routine 140 advances to step 178 where the control circuit 30 is operable to execute a LOT (latch opening time) and oil temperature processing routine for a first transmission oil type, Y. Illustratively, the first transmission oil type, Y, is TES295 or TES389 transmission oil, although the first transmission oil type, Y, may alternatively be set to another conventional transmission oil type. Thereafter at step 180, the control circuit 30 is operable to execute another LOT (latch opening time) and oil temperature processing routine for a second type of transmission oil Y. Illustratively, the second transmission oil type, Y, is C4 transmission oil, although the second transmission oil type, Y, may alternatively be set to another conventional transmission oil type. Those skilled in the art will recognize that the routine 140 may include more or fewer steps such as step 178 and step 180 to thereby execute a LOT (latch opening time) and TMP (transmission oil temperature) processing routine for any number of specified transmission oil types. In any case, after each of the number of LOT and TMP processing routines is complete, the routine 140 advances to step 172 where the routine 140 is returned to the process 100 of FIG. 2.

Referring now to FIG. 4, a flow chart is shown of one illustrative embodiment of the LOT (latch valve opening time) average determination routine used at steps 154, 158, 162, 166 and 170 of the transmission oil type determination routine 140 of FIG. 3. In the illustrated embodiment, the LOT average determination routine 190 begins at step 192 where the latch valve opening time, LOT, is stored in a TX buffer, wherein X is an integer value that may range between 1 and 5 depending upon which of steps 154, 158, 162, 166 and 170 of the transmission oil type determination routine 140 of FIG. 3 is being executed. If, for example, step 166 of the transmission oil type determination routine 140 is being executed, X=4 and the control circuit 30 is operable at step 192 of the LOT average determination routine 190 to store the current value of the latch valve opening time, LOT, in a T4 buffer. Illustratively, each of the TX buffers has a predetermined length, and is configured to store therein only the most recent K values of the latch valve opening time, LOT, that meet the corresponding criteria set forth in steps 152, 156, 160, 164 and 168 of the transmission oil type determination routine 140. In one illustrative embodiment, K equals 5 so that each TX buffer is configured to hold 5 LOT values, although K may alternatively be any positive integer. Following step 192, the control circuit 30 is operable at step 194 to determine what whether the TX buffer is full. Generally, a TX buffer is full if the TX buffer contains K values. If, at step 194, the control circuit 30 determines that the TX buffer contains less than K values, the routine 190 advances to step 196 where the control circuit 30 is operable to set the corresponding latch valve opening time average value, LOT_{AVTX} equal to zero. If, on the other hand, the control circuit 30 determines at step 194 that the TX buffer contains K latch valve opening time values, LOT, the routine 190 advances to step 198 where the control circuit 30 is operable to compute an average of the K latch valve opening time values, LOT, stored in the TX buffer. Illustratively, the control circuit 30 is operable at step 198 to compute the average of the K latch valve opening time values, LOT, stored in the TX buffer in accordance with a conventional algebraic average of the K LOT values, although this disclosure contemplates alternatively computing at step 198 the average of the K latch valve opening time values, LOT, stored in the TX buffer in accordance with other conventional weighted or non-weighted averaging techniques. In any case, both of the steps 196 and step 198 advance to step 200 where the LOT average determination routine 190 is returned to the transmission oil type determination routine 140 of FIG. 3.

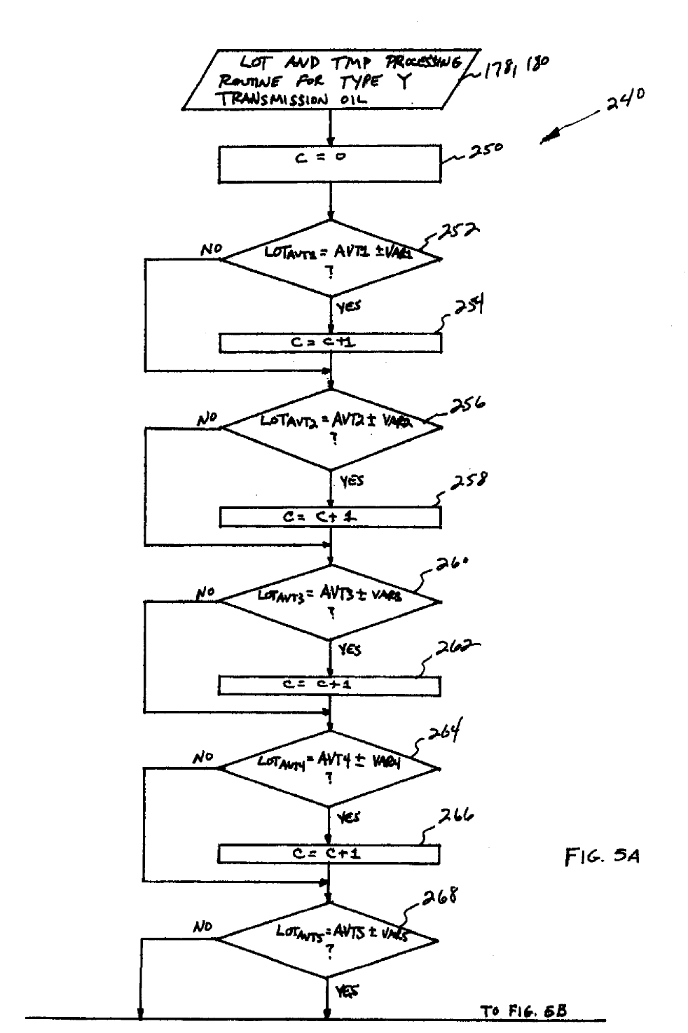

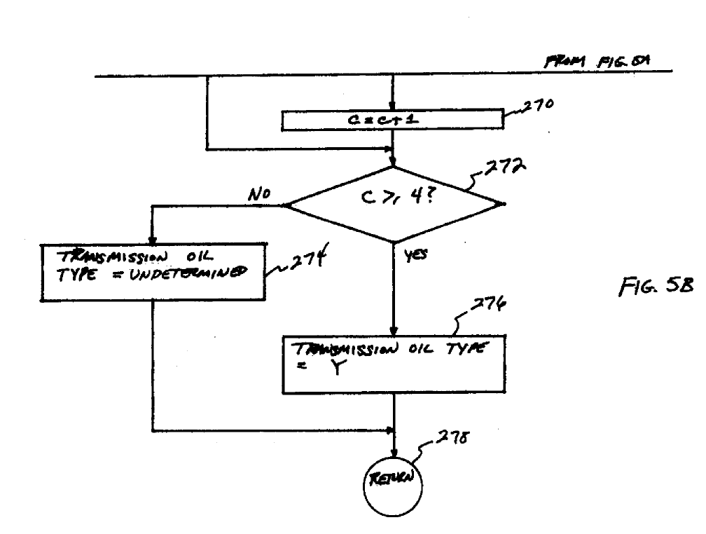

Referring now to FIGS. 5A and 5B, a flow chart of one illustrative embodiment 240 of the LOT (latch valve opening time) and TMP (transmission oil temperature) processing routine executed at steps 178 and 180 of the transmission oil type determination routine 140 of FIG. 3 is shown. Steps 178 and 180 will generally pass the transmission oil type against which the LOT average values will be tested to the routine 240 via the parameter Y. The average temperature and variance values used by the routine 240, as will be described hereinafter, will be defined by the parameter Y.

In the illustrated embodiment, the routine 240 begins at step 250 where the control circuit 30 is operable to set a counter value, C, equal to zero. Thereafter at step 252, the control circuit 30 is operable to determine whether the first average latch valve opening time value, LOT_{AVT1}, determined at step 154 of the transmission oil type determination routine 140 of FIG. 3 is within a first variance value, VAR1, of a first average latch valve opening time value, AVT1. If so, the routine 240 advances to step 254 where the control circuit 30 is operable to increment the count value, C, by 1. From step 254, and from the “NO” branch of step 252, the routine 240 advances to step 256. At step 256, the control circuit 30 is operable to determine whether the second average latch valve opening time value, LOT_{AVT2}, determined at step 158 of the transmission oil type determination routine 140 of FIG. 3 is within a second variance value, VAR2, of a second average latch valve opening time value, AVT2. If so, the routine 240 advances to step 258 where the control circuit 30 is operable to increment the count value, C, by 1. From step 258, and from the “NO” branch of step 256, the routine 240 advances to step 260 where the control circuit 30 is operable to determine whether the third average latch valve opening time value, LOT_{AVT3}, determined at step 162 of the transmission oil type determination routine 140 of FIG. 3 is within a third variance value, VAR3, of a third average latch valve opening time value, AVT3. If so, the routine 240 advances to step 262 where the control circuit 30 is operable to increment the count value, C, by 1. From step 262, and from the “NO” branch of step 260, the routine 240 advances to step 264 where the control circuit 30 is operable to determine whether the fourth average latch valve opening time value, LOT_{AVT4}, determined at step 166 of the transmission oil type determination routine 140 of FIG. 3 is within a fourth variance value, VAR4, of a fourth average latch valve opening time value, AVT4. If so, the routine 240 advances to step 266 where the control circuit 30 is operable to increment the count value, C, by 1. From step 266, and from the “NO” branch of step 264, the routine 240 advances to step 268 where the control circuit 30 is operable to determine whether the fifth average latch valve opening time value, LOT_{AVT5}, determined at step 170 of the transmission oil type determination routine 140 of FIG. 3 is within a fifth variance value, VAR5, of a fifth average latch valve opening time value, AVT5. If so, the routine 240 advances to step 270 where the control circuit 30 is operable to increment the count value, C, by 1. From step 270, and from the “NO” branch of step 260, the routine 240 advances to step 272.

At step 272, the control circuit 30 is operable to determine whether the count value, C, is greater than or equal to 4. If so, the LOT and TMP processing routine 240 has successfully determined a transmission oil type, and the routine 240 advances to step 276 where the control circuit 30 sets the transmission oil type equal to the transmission oil type indicator Y. If, however, the control circuit 30 determines at step 272 that the count value, C, is less than four, then the LOT and TMP processing routine 240 was unable to determine a type of the transmission oil in the transmission 12, and the routine 240 advances to step 274 where the control circuit 30 is operable to set the transmission oil type equal to an undetermined oil type indicator. It will be understood that while a count value, C, of four is used by the routine 240 to distinguish between a determined, i.e., identified, and an undetermined oil type, other values of C may be used. In any case, steps 274 and 276 of the routine 240 advance to step 278 where the LOT and TMP processing routine 240 is returned to the transmission oil type determination routine 140 of FIG. 3.

As described above, steps 178 and 180 will generally pass the transmission oil type against which the LOT average values will be tested to the routine 240 via the parameter Y, and the average time, AVTX, and variance values, VARX, used by the routine 240 (where X is again an integer that ranges, in the illustrated embodiment, between 1 and 5) will be defined by the parameter Y. Generally, each pair of the average time values, ATX, and the corresponding variance values, VARX, correspond to an expected latch valve opening time and corresponding opening time variance value at a corresponding transmission oil temperature. In one example embodiment, the memory unit 32 will have a number of transmission oil maps stored therein, wherein each of the number of maps contains AVTX and VARX values for a different transmission oil type that is identified by the parameter Y. In the embodiment of the transmission oil type determination routine 140 illustrated in FIG. 3, for example, the average LOT data is tested at steps 178 and 180 against two different transmission oil types Y=TES295 and Y=C4. In this example embodiment, the memory unit 32 has two maps in the form of tables stored therein that each contain AVTX and VARX values for a corresponding one of the transmission oil types TES295 and C4. The appropriate table to use during any execution of the routine 240 will be identified by the value of the parameter Y. Table I below illustrates example AVTX and VARX values for Y=TES295 transmission oil, and Table II below illustrates example AVTX and VARX values for Y=C4 transmission oil.

TABLE I
(Y = TES295/TES389 transmission oil)

| X | AVTX (seconds) | VARX (seconds) |
|---|---|---|
| 1 | 0.129 | 0.066 |
| 2 | 0.110 | 0.060 |
| 3 | 0.100 | 0.061 |
| 4 | 0.094 | 0.184 |
| 5 | 0.085 | 0.055 |

TABLE II
(Y = C4 transmission oil)

| X | AVTX (seconds) | VARX (seconds) |
|---|---|---|
| 1 | 0.5 | 0.350 |
| 2 | 0.5 | 0.350 |
| 3 | 0.5 | 0.450 |
| 4 | 0.5 | 0.450 |
| 5 | 0.5 | 0.450 |

It will be understood that the values in Table I and Table II are provided only by way of example, and other data values may be used for these and/or other transmission oil types. Each set of the AVTX and VARX values will, however, correspond in any case to the base temperature values, e.g., T1-T5, used in the execution of the transmission oil type determination routine 140 of FIG. 3. In other words, AVT1 and VAR1 will be selected to be appropriate average time and variance values for the corresponding transmission oil in the temperature range T1+/−J°, AVT2 and VAR2 will be selected to be appropriate average time and variance values for the corresponding transmission oil in the temperature range T2+/−J°, and so forth. It will also be understood that more or fewer such tables may be stored in the memory 32 to set forth AVTX and VARX values for correspondingly more or fewer transmission oil types. It will still further be understood that any such AVTX and VARX information may alternatively be stored in the memory unit 32 in the form of one or more plots, graphs, models or the like.

While the invention has been illustrated and described in detail in the foregoing drawings and description, the same is to be considered as illustrative and not restrictive in character, it being understood that only illustrative embodiments thereof have been shown and described and that all changes and modifications that come within the spirit of the invention are desired to be protected.

What is claimed is:

1. A system for determining transmission oil type, the system comprising:
a transmission including a latch valve positioned between an oil reservoir containing transmission oil and one or more torque transmitting devices,
an actuator configured to be responsive to an actuator control signal to open the latch valve to supply oil from the oil reservoir to the one or more torque transmitting devices,
a first sensor configured to produce a sensor signal from which an opening time of the latch valve can be determined,
a second sensor configured to produce a temperature signal corresponding to a temperature of the oil supplied by the latch valve to the one or more torque transmitting devices, and
a control circuit including a memory having instructions stored therein that are executable by the control circuit to process the sensor and temperature signals, following production of the actuator control signal, over a range of oil temperatures to collect opening times of the latch valve at each of a plurality of temperatures within the range of oil temperatures, and to then determine the transmission oil type based on the opening times of the latch valve at each of the plurality of temperatures.

2. The system of claim 1 further comprising an oil pump positioned between the latch valve and the oil reservoir, the oil pump configured to be responsive to a pump control signal to supply oil from the oil reservoir to the latch valve,
wherein the control circuit is configured to produce the pump control signal.

3. The system of claim 2 wherein the latch valve defines an inlet fluidly coupled to the oil pump and an outlet fluidly coupled to the one or more torque transmitting devices,
and wherein the first sensor comprises a pressure sensor configured to produce a pressure signal corresponding to a pressure of the oil exiting the outlet of the latch valve.

4. The system of claim 3 wherein the instructions stored in the memory further include instructions that are executable by the control circuit to monitor the pressure signal following production of the actuator control signal and to determine that the latch valve is open when the pressure of the oil exiting the outlet of the latch valve reaches a predefined pressure value.

5. The system of claim 1 wherein the latch valve defines an inlet configured to receive oil from the oil reservoir and an outlet configured to supply the oil received from the oil reservoir to the one or more torque transmitting devices,
and wherein the first sensor comprises a position sensor configured to produce a position signal corresponding to a position of the latch valve relative to a reference position.

6. The system of claim 5 wherein the instructions stored in the memory further include instructions that are executable by the control circuit to monitor the position signal following production of the actuator control signal and to determine that the latch valve is open when the position of the latch valve relative to the reference position reaches a predefined position.

7. The system of claim 1 wherein the instructions stored in the memory further include instructions that are executable by the control circuit to process the sensor and temperature signals, following production of the actuator control signal over the range of oil temperatures, to collect a plurality of opening times of the latch valve at each of a plurality of temperatures within the range of oil temperatures, to process the plurality of opening times of the latch valve at each of the plurality of temperatures to determine an average opening time of the latch valve at each of the plurality of temperatures, and to then determine the transmission oil type based on the average opening times of the latch valve at each of the plurality of temperatures.

8. The system of claim 1 further comprising a transmission oil map stored in memory, the transmission oil map defining expected opening time values and corresponding opening time variance values at each of the plurality of temperatures for a predefined transmission oil type,
and wherein the instructions stored in the memory further include instructions that are executable by the control circuit to compare the opening times of the latch valve to the expected opening time values and corresponding opening time variance values at each of the plurality of temperatures, and to determine that the transmission oil is the predefined transmission oil type if differences between at least a predefined number of the opening times of the latch valve match corresponding ones of the expected opening time values within the corresponding opening time variance values.

9. The system of claim 1 further comprising a plurality of transmission oil maps stored in memory, each of the plurality of transmission oil maps defining expected opening time values and corresponding opening time variance values at each of the plurality of temperatures for a different transmission oil type,
and wherein the instructions stored in the memory further include instructions that are executable by the control circuit to compare the opening times of the latch valve to the expected opening time values and corresponding opening time variance values at each of the plurality of temperatures for each of the plurality of transmission oil maps, and to determine that the transmission oil is a transmission oil type defined by one of the plurality of transmission oil maps if differences between at least a predefined number of the opening times of the latch valve match corresponding ones of the expected opening time values of the one of the plurality of transmission oil maps within the corresponding opening time variance values.

10. A method for determining transmission oil type, the method comprising:
opening a latch valve positioned between an oil reservoir and one or more torque transmitting devices of a transmission to supply transmission oil from the reservoir to the one or more torque transmitting devices,
determining an opening time of the latch valve,
determining a temperature of the oil supplied by the latch valve to the one or more torque transmitting devices in response to opening the latch valve,
repeatedly opening the latch valve, determining an opening time of the latch valve and determining a temperature of the oil supplied by the latch valve over a range of oil temperatures to collect opening times of the latch valve at each of a plurality of oil temperatures within the range of oil temperatures,
determining the transmission oil type based on the opening times of the latch valve at each of the plurality of oil temperatures, and
storing the transmission oil type in memory.

11. The method of claim 10 further comprising:
collecting a plurality of opening times of the latch valve at each of a plurality of temperatures within the range of oil temperatures, and
processing the plurality of opening times of the latch valve at each of the plurality of temperatures to determine an average opening time of the latch valve at each of the plurality of temperatures,
and wherein determining the transmission oil type comprises determining the transmission oil type based on the average opening times of the latch valve at each of the plurality of temperatures.

12. The method of claim 10 further comprising comparing the opening times of the latch valve to expected opening time values and corresponding opening time variance values for a predefined transmission oil type at each of the plurality of temperatures, and
and wherein determining the transmission oil type comprises determining that the transmission oil is the predefined transmission oil type if differences between at least a predefined number of the opening times of the latch valve match corresponding ones of the expected opening time values within the corresponding opening time variance values.

13. The method of claim 10 further comprising comparing the opening times of the latch valve to expected opening time values and corresponding opening time variance values at each of the plurality of temperatures for each of a plurality of different transmission oil types, and
and wherein determining the transmission oil type comprises determining that the transmission oil is a transmission oil type defined by one of the plurality of different transmission oil maps if differences between at least a predefined number of the opening times of the latch valve match corresponding ones of the expected opening time values of the one of the plurality of different transmission oil maps within the corresponding opening time variance values.

14. The method of claim 10 further comprising storing an undetermined oil type indicator in memory if the transmission oil type cannot be determined based on the opening times of the latch valve at each of the plurality of oil temperatures.

15. The method of claim 10 further comprising activating a warning indicator if the transmission oil type cannot be determined based on the opening times of the latch valve at each of the plurality of oil temperatures.

16. The method of claim 10 further comprising, if the transmission oil type cannot be determined based on the opening times of the latch valve at each of the plurality of oil temperatures, logging at least one of miles traveled by a vehicle carrying the transmission and operating time of the transmission following determination that the transmission oil type cannot be determined.

17. The method of claim 10 further comprising modifying an oil change scheduling process if the transmission oil type cannot be determined based on the opening times of the latch valve at each of the plurality of oil temperatures.

18. The method of claim 10 further comprising activating a warning indicator if the transmission oil type is determined, based on the opening times of the latch valve at each of the plurality of oil temperatures, to be an undesirable transmission oil type.

19. The method of claim 10 further comprising, if the transmission oil type is determined to be an undesirable transmission oil type based on the opening times of the latch valve at each of the plurality of oil temperatures, logging at least one of miles traveled by a vehicle carrying the transmission and operating time of the transmission following determination that the transmission oil type is undesirable.

20. The method of claim 10 further comprising modifying an oil change scheduling process if the transmission oil type is determined, based on the opening times of the latch valve at each of the plurality of oil temperatures, to be an undesirable transmission oil type.

21. A method for determining transmission oil type, the method comprising:
determining an opening time of a latch valve positioned between an oil reservoir and one or more torque transmitting devices of a transmission in response to a command to open the latch valve,
determining a temperature of oil supplied by the latch valve from the oil reservoir to the one or more torque transmitting devices in response to the command to open the latch valve,
repeating determining an opening time of the latch valve and determining a temperature of oil supplied by the latch valve over a range of oil temperatures to collect opening times of the latch valve at each of a plurality of oil temperatures within the range of oil temperatures,
determining the transmission oil type based on the opening times of the latch valve at each of the plurality of oil temperatures, and
storing the transmission oil type in memory.
